(19) United States
(12) Patent Application Publication
(10) Pub. No.: US 2009/0042782 A1
(43) Pub. Date: Feb. 12, 2009

(54) Diagnostic and therapeutic strategies for conditions associated with the FLJ13639 gene

(76) Inventors: Lionel J. Coignet, East Amherst, NY (US); Ari-Nareg Meguerditchian, Buffalo, NY (US); Alain Nganga, Buffalo, NY (US); Timothy Johnson, Panama, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 11/635,979

(22) Filed: Dec. 7, 2006

Related U.S. Application Data
(60) Provisional application No. 60/748,652, filed on Dec. 8, 2005.

(51) Int. Cl. A61K 38/16; G01N 33/68; G01N 33/48; C12Q 1/68; A61P 35/00; G01N 33/53; C07K 14/00

(52) U.S. Cl. 514/12; 436/86; 436/94; 435/6; 435/7.92; 530/350

(57) ABSTRACT

Provided are methods for determining a prognosis for an individual suspected of having or diagnosed with cancer, where a high CD24/low FLJ13639 ratio indicates an unfavorable prognosis. Also provided are methods for enhancing the activity of chemotherapeutic agents, and isolated, purified FLJ13639 proteins for use in improving the prognosis of an individual and for enhancing the activity of chemotherapeutic agents.

This application claims priority to U.S. provisional application No. 60/748,652, filed on Dec. 8, 2005, the disclosure of which is incorporated herein by reference.

### FIELD OF THE INVENTION

This invention relates generally to the field of prognostic indicators for hematological malignancies and solid tumors, and treatments therefor using proteins of the invention.

### DESCRIPTION OF RELATED ART

The 13q14 region is frequently altered in several hematological malignancies as well as solid tumors. Examples include but are not limited to prostate, breast, and colorectal cancers. 13q14 alterations, mainly deletions, are frequently observed in acute leukemia (1), multiple myeloma and mantle cell lymphoma. Alterations of 13q14 (seen in 25-40% of samples) constitute a statistically significant independent adverse prognostic factor for myeloma patients (2-4). It has also been shown that deletion of chromosome 13 is associated with transition from Monoclonal Gammopathy of Unknown Significance (MGUS) to multiple myeloma (5). Deletion of 13q14 has been also observed in 50% of mantle cell lymphoma cases (6). Similarly, 13q14 alterations have been described for a variety of solid tumors. However, genes associated with 13q14 chromosomal alterations and the relationship of such alterations with malignant phenotypes are poorly characterized. Further, while certain established prognostic parameters for malignancies associated with 13q14 alterations are known, they are limited in predicting the behavior of the malignancies in individual patients. Thus, there is a need for patient-specific information for use in individualized prognostic assessment for persons having malignancies associated with 13q14 alterations.

### SUMMARY OF THE INVENTION

In the present invention a method is provided for determining the prognosis of an individual diagnosed with or suspected of having cancer. The method comprises obtaining a biological sample from the individual and assaying the sample to determine a ratio of CD24 expression to FLJ13639 expression. A high CD24/low FLJ13639 expression level is predictive of an unfavorable prognosis.

In another embodiment, a low FLJ13639 expression level relative to a normal, non-malignant cell is predictive of an unfavorable prognosis.

Also provided are isolated, purified FLJ3639 proteins, such as recombinant FLJ3639 proteins, and a method of using the proteins for improving the prognosis of an individual who is diagnosed with or suspected of having cancer and who exhibits a high CD24/low FLJ13639 expression level profile. The method of improving the prognosis of the individual comprises administering a recombinant FLJ3639 protein to the individual in an amount effective to improve the prognosis of the individual. In one embodiment, the prognosis of the individual is improved by reducing the proliferation and/or metastasis of cancer cells in the individual.

The invention also provides a method for enhancing the effect of a chemotherapeutic agent. This method comprises administering a chemotherapeutic agent in combination with an amount of FLJ3639 protein effective to enhance the effect of the chemotherapeutic agent.

### BRIEF DESCRIPTION OF THE DRAWINGS

FIGS. 1A and 1B are photographic representations of RT-PCR results for FLJ13639 and WDFY2 genes, respectively in the MUTZ5 cell line, as well as for G519 and EBN-Lin cell lines as controls.

FIG. 2 is a schematic representation of the three FLJ13639 transcripts and their respective open reading frames.

FIG. 3 is a photographical representation of the cellular localization of FLJ13639 P1, P2 and P3. All three show mitochondrial localization with a same pattern as Mitotracker, a specific marker of mitochondria. Selective sub-cloning of the first 20 amino acids or the remaining amino acids of the FLJ13639 protein demonstrates the role of the first 20 amino acids for P1's mitochondrial localization.

FIG. 4A is a photographic representation of electrophoretic separation of RT-PCR products representing CD24 and FLJ13639/P1 expression in a series of leukemia cell lines and controls.

FIG. 4B. is a photographic representation of electrophoretic separation of RT-PCR products for CD24/FLJ13639/P1 from four ovarian cancer cell lines and EBV-LIN (a lymphoblastoid cell line). Of note, there is an inversion of profile between the parental A2780 and the cisplatin resistant derived cell line A2780CP3 (as well as with the other resistant cell lines) which shows that a shift from a high CD24/low FLJ13639 expression profile is linked with chemoresistance.

FIG. 4C is a photographic representation of electrophoretic separation of RT-PCR products representing CD24 and FLJ13639 expression in three lung cancer cell lines.

FIG. 4D is a photographic representation of electrophoretic separation of RT-PCR products representing CD24 and FLJ13639 expression in prostate cancer cell lines.

FIG. 4E is a photographic representation of electrophoretic separation of RT-PCR products representing CD24 and FLJ13639 expression in a series of invasive breast cancer cell lines.

FIG. 5A is a photographic representation of electrophoretic separation of RT-PCR products representing CD24 and FLJ13639 expression in biological samples obtained from leukemia patients.

FIG. 5B is a photographic representation of electrophoretic separation of RT-PCR products representing CD24 and FLJ13639 expression in biological samples obtained from ovarian cancer patients.

FIG. 5C is a photographic representation of electrophoretic separation of RT-PCR products representing CD24 and FLJ13639 expression in biological samples obtained from lung cancer patients.

FIG. 6 provides a Kaplan-Meier survival curve based on the classification of 29 patient samples tested for CD24 and FLJ13639/P1 expression by RT-PCR.

FIG. 7A is a photographic representation of electrophoretic separation of RT-PCR amplification of CD24 and FLJ13639/P1 cDNA from clinical samples of breast cancer tumors. Upper left panel: no staining (0); upper right panel: low staining (1+); lower left panel: intermediate staining (2+); lower right panel: intense staining (3+).

FIG. 7B is an illustration of CD24 staining on paraffin embedded invasive breast cancer tumors included in a tissue macro array. The tissue sections were stained with a commercially available anti-CD24 antibody to assess the correlation between CD24 staining and stage of the disease. Advanced disease is associated with greater CD24 staining.

FIG. 8A is a representation of the pTAT-HA-FLJ13639/P1 vector.

FIG. 8B is a photographic representation of SDS-PAGE separation of the FLJ13639/P1-TAT recombinant protein.

FIG. 9A is a graphical representation of the alteration of CD24 mRNA expression upon incubation of UoCB1 cells with increasing amount of FLJ13639/P1-TAT recombinant protein.

FIG. 9B is a graphical representation of the alteration of CD24 expression upon incubation of lung cancer cell lines with FLJ13639/P1-TAT.

FIG. 10A is a photographic representation of alteration of UoCB1 cells invasiveness potential upon treatment with FLJ13639/P1-TAT recombinant protein (right panel) or control (left panel).

FIG. 10B is a graphical summary of reduction of invasiveness upon treatment with P1-TAT shown in FIG. 10A.

FIG. 11A is a graphical representation of a time-course analysis of CD24 expression level in UoCB1 cells after 6, 12, 18 and 24 hour incubations with 20 μg of FLJ13639/P1-TAT.

FIG. 11B provides photographic representations of CD24 expression assessed by immunofluorescence in the OM9;22 ALL cell line with or without P1-TAT treatment for 48 hours.

FIG. 12A is a photographic representation of a wound healing assay performed on lung cancer cell lines. The H520 cells were treated (or not treated) with the FLJP1-TAT and motility of cells was monitored through a light microscope to analyze closing of the created gap. As can be seen in this figure, the gap is almost completely filled after 5 days whereas the FLJ13639P1-TAT treatment inhibited filling of the gap, which is indicative of inhibited motility and cell proliferation.

FIG. 12B is a wound healing assay as depicted in FIG. 12A performed on breast cancer cells.

FIG. 13 is a graphical representation of lactic acid production in HeLa cells with or without treatment with the FLJ13639P1-TAT recombinant protein. * indicates statistical significant difference.

FIG. 14A is a graphical representation of green/red fluorescence intensity ratio data measured for treatment of HL60/VDR cells (a vinctrisin resistant cell line) as indicated for the x-axis. * indicates statistical significance.

FIG. 14B is a photographic representation of cells exhibiting a low or a high green/red ratio, indicating a non-disrupted mitochondrial membrane potential (left panel), whereas the predominantly green staining (right panel) indicates a disrupted mitochondrial membrane potential.

FIG. 15 is a graphical representation of the results from viability assay with HL60/VCR upon treatment with sub-optimal dose of vincristin with or without FLJ13639/P1-TAT recombinant protein.

FIG. 16 is a photographic representation of a Western blot using FLJ13639/P1-TAT MAb.

### DETAILED DESCRIPTION OF THE INVENTION

The present invention provides a method of determining the prognosis of an individual diagnosed with or suspected of having cancer. The method comprises obtaining a biological sample from the individual and assaying the sample to determine a ratio of CD24 expression to FLJ13639 expression, wherein a high CD24/low FLJ13639 expression level is predictive of an unfavorable prognosis. In another embodiment, a low FLJ13639 expression level alone relative to a normal cell is predictive of an unfavorable prognosis

Also provided is a method for identifying an individual as a candidate for therapy with FLJ13639 protein. The method comprises obtaining a biological sample from the individual and assaying the sample to determine the ratio of CD24 expression to FLJ13639 expression, wherein a high CD24/low FLJ13639 expression ratio identifies the individual as a candidate for therapy with FLJ13639 protein.

In another embodiment the invention provides isolated, purified FLJ13639 proteins and compositions comprising the same, as well as a method of using such compositions for improving the prognosis of an individual diagnosed with or suspected of having cancer. The method comprises administering to the individual an amount of FLJ13639 protein sufficient to improve the prognosis of the individual. It is considered that the prognosis of the individual will be improved by reducing the proliferation and/or metastasis of cancer cells in the individual.

In yet another embodiment, a method is provided for enhancing the effect of a chemotherapeutic agent. The method comprises administering a chemotherapeutic agent in combination with an amount of FLJ13639 protein effective to enhance the effect of the chemotherapeutic agent. It is demonstrated that this method is effective for enhancing the activity of a chemotherapeutic agent against cells resistant to the chemotherapeutic agent.

FLJ13639 is also referred to as “DHRS12.” Thus, for the purposes of this description, reference to a DHRS12 database entry is synonymous with reference to the FLJ13639 gene and/or the mRNA and protein products which result from expression of the FLJ13639 gene.

We have determined that the FLJ13639 gene is expressed as at least three distinct transcript sizes, referred to herein as “P1” “P2” and “P3”. Of these, and without intending to be bound by any particular theory, P1 is thought to be the active form of FLJ13639. The sequence of the cDNA encoding P1 is provided here as SEQ ID NO:1. The DNA sequence of the cDNA encoding P2 is provided here as SEQ ID NO:3. The sequence of the cDNA encoding P3 is provided here as SEQ ID NO:4. It will be apparent to those skilled in the art that the amino acid sequences of the P2 and P3 proteins can be deduced from their respective cDNA sequences. While the same applies to the P1 protein, for convenience, its amino acid is provided here as SEQ ID NO:2.

The FLJ13639 gene lies within the human chromosome 13 genomic contig reference assembly, available under GenBank accession number NT_{—}024524, Aug. 26, 2006 entry. When determining FLJ13639 expression in accordance with the present invention, it is preferable to measure either the amount of P1 mRNA or the amount of protein expressed from P1 mRNA.

CD24 is a small, mucin-like glycosylated protein core of 27 amino acids (Lim S C, Biomed Pharmacother. (2005) Vol: 59 (Suppl 2): S351-4). Its gene is localized on chromosome 6q21 (Kristiansen G, et al. J Molec Histol 2004 March; 35(3): 255-262). CD24 serves as a specific ligand for P-selectin (Aigner S, et al. Blood. (1997) Vol. 89(9): 3385-95). The latter is found on activated platelets and endothelial cells, and mediates adhesion and rolling by interacting with specific sialylated carbohydrates (Lim S C). Without intending to be bound by any particular theory, it is considered that CD24 positive tumor cells may possess an increased propensity for cell adhesion that precedes the growth of new metastasis, and that this pathway may be an important element in recruiting and exiting tumor cells towards target organs. The sequence of the CD24 cDNA provided as SEQ ID NO:6.

The present invention facilitates the determination of a prognosis and/or the improvement of the prognosis of individuals diagnosed with or suspected of having any of a variety of malignancies. For example, such malignancies may include solid tumors (such as prostate, breast, colorectal, lung (small cell and non-small cell), ovarian, melanoma, urinary system, uterine, endometrial, pancreatic, oral cavity, thyroid, stomach, brain and other nervous system, liver, and esophagial) or hematological cancers, such as Hodgkins Lymphoma, Non-Hodgkins Lymphoma (NHL), chronic and other leukemias, and myeloma.

Biological samples suitable for use in the present invention can be obtained via routine methods. The selection of a biological sample source for use in the invention is dictated by the type of cancer the individual is suspected of having or has been diagnosed with. For example, for leukemias, it is preferable to determine the expression ratio of FLJ13639 and CD24 from a sample of bone marrow. However, for solid tumors, a biopsy of the tumor is preferable.

The ratio of CD24/FLJ13639 expression can be determined by analyzing mRNA or protein from a biological sample using any suitable method. The mRNA or protein can be measured from different samples, or from different portions of the same sample. However, as will be appreciated by those skilled in the art, it is preferable for the CD24/FLJ13639 ratio to be obtained by comparing the FLJ13639 mRNA level determined for a particular biological sample to the CD24 mRNA level determined for the same biological sample, or by comparing the FLJ13639 protein level determined for a particular biological sample to the CD24 protein level determined for the same sample.

Any suitable method for determining the level of FLJ13639 and CD24 mRNA expression can be used. For example, mRNA levels can be determined by standard techniques, including Northern blotting, slot blotting, ribonuclease protection, quantitative or semi-quantitative RT-PCR, or microarray analysis. Suitable primers or probes for use in any of these techniques can be prepared by standard methods based upon the sequences of FLJ13639 and CD24 cDNA provided herein and in connection with factors affecting nucleic acid hybridization parameters which are well know to those skilled in the art.

CD24 and/or FLJ13639 protein levels can be determined by any acceptable method. Preferred methods include immunodetection methods. For example, Western blots, in situ hybridizations, immuohistochemistry, ELISA, etc. can be used to detect the presence and amount of FLJ13639 and CD24 proteins. In this regard, the present invention also provides monoclonal antibodies (mAbs) for use in detecting FLJ13639 protein and measuring FLJ13639 protein expression levels. We have prepared FLJ13639 mAbs which include mAbs 4B3, 4B4, 1A9, 2C12, 9A11, 4E8, 10F11, 2F5, 10D1, 4G7, 8E1, 8H9. Such mAbs could be used, for example, to detect the ratio of CD24/FLJ13639 protein levels by flow cytometry analysis of a biological sample, or for immunostaining, such as in immunocytohistchemical analysis of paraffin embedded or frozen tumor samples.

The present invention also provides isolated, purified FLJ13639 proteins, such as recombinant FLJ13639 proteins. Recombinant FLJ13639 proteins can be prepared using any suitable methods. In one embodiment, a protein translation expression vector comprising the FLJ13639 PI cDNA can be introduced into an appropriate host cell from which recombinant protein can be produced and extracted using conventional methods, such as those described in Sambrook, et al., MOLECULAR CLONING: A LABORATORY MANUAL, 2nd Ed., Cold Spring Harbor Laboratory Press, Cold Spring Harbor, N.Y. (1989). It will also be recognized that routine modifications to the protein sequence for the purposes of facilitating protein purification, such as by incorporating conventional protein purification tags, or to improve the pharmacological delivery of the protein are within the purview of those skilled in the art. For example, the FLJ13639 protein can be modified so as to comprise a transduction domain of a protein, such as the HIV-1 TAT domain, to facilitate passage of the proteins through biological membranes independent of classical receptor or endocytosis mediated pathways. Such modified proteins are also considered herein to be recombinant FLJ13639 proteins. It is also contemplated that a nucleic acid encoding FLJ13639 can be used as a therapeutic agent by administering such a nucleic acid to an individual using known gene therapy methods.

Recombinant FLJ13639 proteins can be administered in a conventional dosage form prepared by combining the protein with standard pharmaceutically acceptable carriers. Acceptable pharmaceutical carriers for use with proteins are described in Remington's Pharmaceutical Sciences (18th Edition, A. R. Gennaro et al. Eds., Mack Publishing Co., Easton, Pa., 1990).

Various methods known to those skilled in the art may be used to administer recombinant FLJ13639 protein formulations to an individual. These methods include, but are not limited to, intradermal, intramuscular, intratumoral, intraperitoneal, intravenous, subcutaneous, and intranasal routes. It will be recognized that the dosage of FLJ13639 protein to be used is determined based upon well-known variables, such as the route of administration, the size of the individual and the stage of the disease.

When used in the present method for enhancing the effect of a chemotherapeutic agent, recombinant FLJ13639 protein can be administered prior to, concurrent with, or subsequent to administration of the chemotherapeutic agent. Examples of suitable chemotherapeutic agents include but are not limited to vincristin, doxorubicin and cisplatin. In another embodiment, administration of recombinant FLJ13639 protein can be performed to enhance the anti-cancer effects of radiation therapy and/or photodynamic therapy (PDT).

It will be apparent to those skilled in the art from the following Examples that we have characterized FLJ13639 as a putative dehydrogenase gene located on chromosome 13q14 that is disrupted by a t(12; 13) chromosomal translocation. We have demonstrated that one of the consequences of the loss or reduction of FLJ13639 expression is the over-expression of CD24 and that the ratio of FLJ13639 and CD24 expression is prognosticative of patient survival. We have developed an FLJ13639 recombinant protein and showed that restoration of FLJ13639 levels with this recombinant protein induces down-regulation of CD24, reduces or blocks invasion and motility of cancer cells, inhibits their growth and enhances chemosensitivity in cells that exhibit a high CD24/low FLJ13639 profile. We have also demonstrated that the reintroduction of the FLJ13639 protein restores essential cell functions that are associated with the mitonchondria. However, these Examples are meant to illustrate, but not to limit the invention.

### EXAMPLE 1

This Example demonstrates the characterization of 13q breakpoints in acute leukemia (1). To facilitate this characterization, we derived a cell line from a patient sample harboring a unique t(12;13)(p12;q14) translocation (7). We used this cell line to identify the gene(s) involved in this rearrangement to establish a molecular characterization of the pathogenetic events potentially leading to the occurrence of leukemia and its persistence, but in addition to hematologic malignancies, this leukemia model is relevant to a wide array of solid tumors as well.

We originally identified the gene involved in a single translocation on chromosome 12p12 as being the ETV6 gene that has been involved in many balanced translocations (1). We attempted to clone the partner gene using molecular RACE techniques were unsuccessful. We therefore undertook a FISH walking approach on chromosome 13 using a series of commercially available RPCI BAC clones which allowed us to identify a BAC clone spanning the translocation breakpoint (RPCI BAC clone RP11-147H23; GenBank Accession no. AL136525, May 18, 2005 entry). Therefore, the sequence for this BAC clone was analyzed to identify which (if any) genes were present in this region. Only two genes were identified: WDFY2, named for its homology to another gene, WDFY1, and a predicted gene, FLJ13639.

To determine which of these genes was altered by the t(12;13) translocation, RT-PCR was performed essentially as described in Example 6 on the MUTZ-5 cell line, with EBV-LIN, a lymphoblatoid cell line as a normal control. Briefly, total RNA was extracted from the cell cultures using conventional techniques and reverse transcription was performed. The primers used in the RT-PCR reaction for WDFY2 have the forward sequence 5′ TCTGTCTCAACCTGTGTCCC 3′ (SEQ ID NO:7) and the reverse sequence 5′ GAAGAGTCCCCTTGCGAGT 3′ (SED ID NO:8). To amplify FLJ13639 P1, a forward primer having the sequence 5′CATCCGGGAGAGCGGTAACC3′ (SEQ ID NO:9) and a reverse primer having the sequence 5′AGCACAGGGATCAGGCCGGTC3 (SEQ ID NO:10) were used.

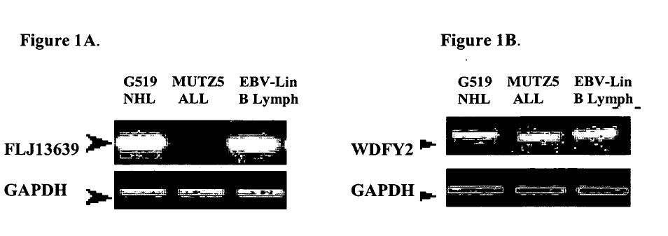

The results indicate WDFY2 displays a normal expression in both cell lines (FIG. 1B) whereas no expression for FLJ13639 is detected in MUTZ-5 (FIG. 1A). (G519 is a cell line which was established from a Non-Hodgkin's lymphoma (NHL) cell line). Thus, the t(12;13) chromosomal disruption results in abrogation of FLJ13639 expression.

### EXAMPLE 2

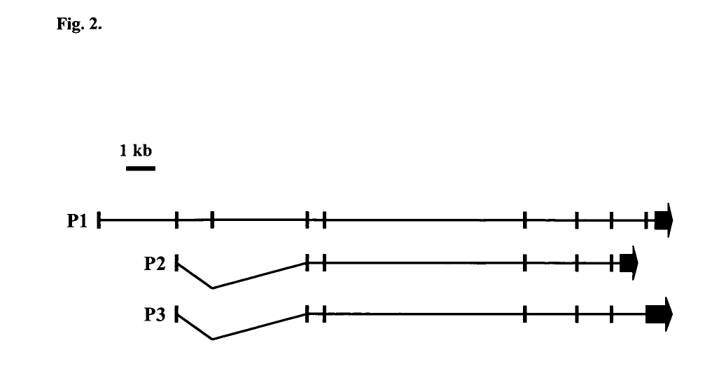

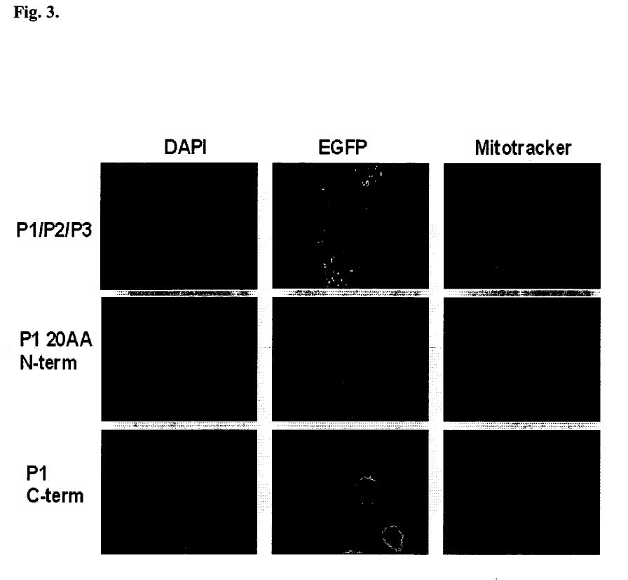

This Example provides a characterization of the FLJ13639 gene. We have determined by bioinformatics analysis that FLJ13639 has homology with the short-chain dehydrogenase reductase super family (SDR). The FLJ13639 protein possesses the classical signatures of SDR, including the common GlyXXXGlyXGly pattern representing the NAD(H) or NADP(H) co-enzyme binding motif, AsnAsnAsnAlaGly (SEQ ID NO:11) folding motif as well as the TyrXXXLys segment representing a suggested substrate binding pocket and a catalytic site (11). We elaborated the three mRNA variants of FLJ13639 (P1, P2 and P3) by bioinformatics analysis using alignments of EST sequences and determined that the three transcripts result from alternative splicing in the 5′ and 3′ end of the gene (summarized in FIG. 2). These three transcripts give rise to three proteins (P1, P2 and P3) which are demonstrated to localize to the mitochondria (FIG. 3). In particular, FIG. 3 illustrates that FLJ13639 Proteins, when expressed as an enhanced green fluoresecent protein fusion (EGFP in FIG. 3) localize to the mitochondria. The sub-cloning of the first 20 N-terminal amino acids (alone as a EGFP fusion protein (“P1 20 AA N-term”; FIG. 3) confirms that the mitochondrial targeting sequence is this region of P1 since its expression pattern is similar to that obtained with the entire FLJ13639-GFP fusion protein. However, when the rest of the protein is expressed with GFP, without the first 20 AA (“P1—Cterm”), the mitochondiral signature is lost. Without intending to be bound by any particular theory, it is considered that P2 and P3 may act on P1 as retrocontrol loops, or “gatekeepers.”

### EXAMPLE 3

To analyze the function of FLJ13639, P1 was subcloned in an antisense orientation in a pCDNA3 expression vector (available from Invitrogen®) and this construct was transfected into immortalized fibroblasts (as well as empty vector as control) using the calcium-phosphate method. The intent was to assess which genes are affected directly or indirectly by the down-regulation of FLJ13639. After transfectant selection, total RNA was extracted from these cells and amplified as cDNA using reverse transcriptase. The cDNAs were analyzed using the Human Affymetrix U133 array. Expression profiles were compared between cells transfected with either vector alone or antisense constructs. Down regulation of FLJ13639 by the antisense assay was confirmed by RT-PCR.

A majority of genes were down regulated (223 down-regulated for 14 up-regulated). We determined that one of the overexpressed genes is CD24, and that it is over-expressed (by 5 fold) when FLJ13639 expression is lost.

### EXAMPLE 4

This Example demonstrates that decreased expression of FLJ13639 is correlated with increased expression of CD24 in leukemia, ovarian, lung, breast and prostate cancer.

The expression levels for CD24 and FLJ13639 were assessed by RT-PCR in acute leukemia cell lines of B-cells (UoCB1, ALL-1, OM9;22, ALLCJ), T-cell (207/CEM) origins, as well as CML blast crisis (K562). The EBV-LIN cell line was also included as control. Determination of CD24 and FLJ13639 expression levels was performed by RT-PCR essentially as described in Example 6. The RT-PCR amplifications generated amplicons of 300 and 233 bp for CD24 and FLJ13639P1, respectively. These amplification products were elaborated by electrophoretic separation in agarose gels and visualized with Ethidium Bromide.

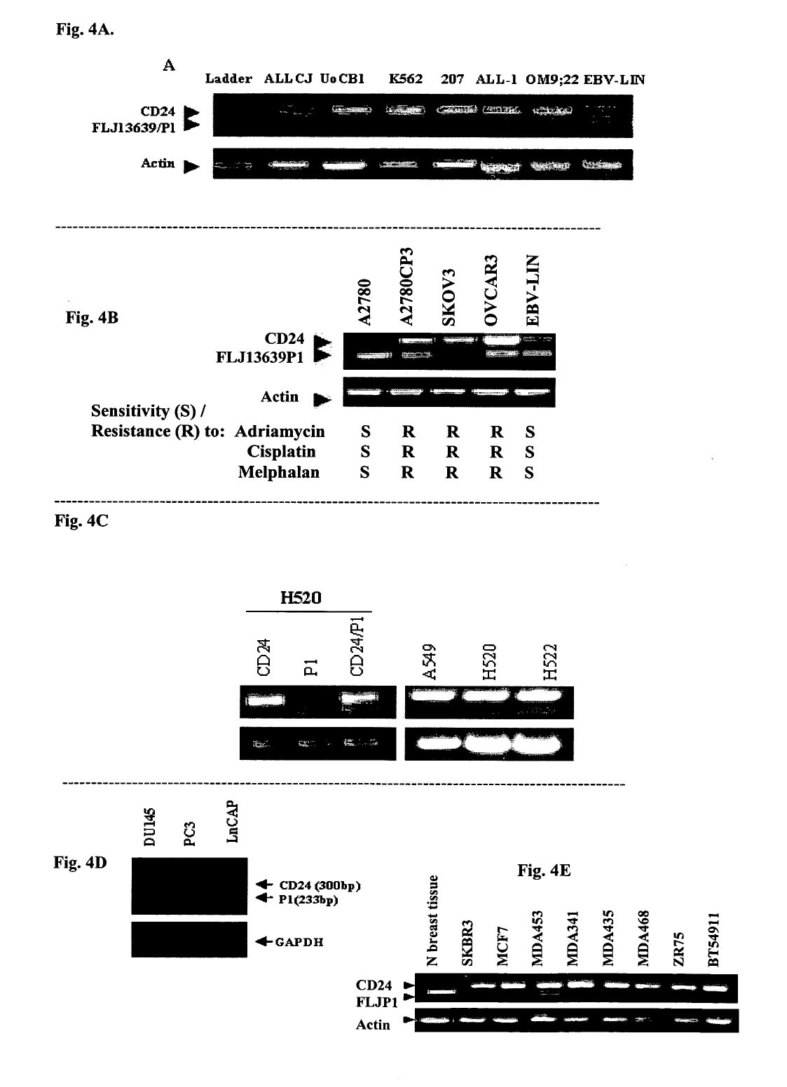

The lymphoblastoid cell line (EBV-LIN) showed a moderate expression of both CD24 and FLJ13639, whereas all the other leukemia cell lines tested were found to have high CD24/low FLJ13639P1 (FIG. 4A). Cell lines from other tumors (ovarian, lung, breast) were tested and showed the same high CD24/low FLJ13639P1 profile (FIGS. 4B-C). The cell lines presented are derived from leukemia (ALLCJ, UOCB1, K562, ALL-1, OM9;22) ovarian cancer (A2780, A2780CP3, SKOV3, OVCAR3); lung cancer (A549, H520, H522); prostate cancer (DU145, PC3, LNCAP); and breast cancer (SKBR3, MCF7, MDA453, MDA341, MDA435, MDA468, ZR75, BT54911). A similar result was obtained for prostate (FIG. 4D) and breast cancer (FIG. 4E). Of note, FIG. 4B demonstrates a shift from a high CD24/low FLJ13639 expression profile to be linked with chemoresistance, since A2780, upon becoming the cisplatin resistant A2780CP3 cell line, exhibits a high CD24/low FLJ13639 profile, in contrast to A2780.

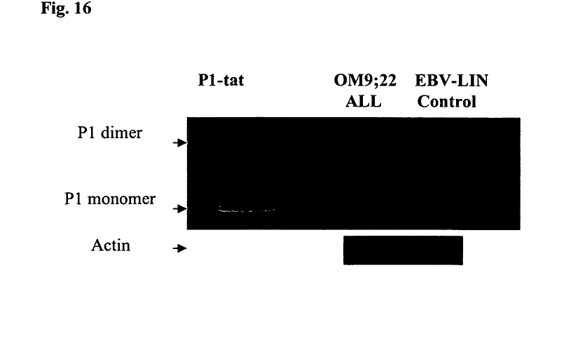

We have also developed monoclonal antibodies to against FLJ13639/P1 and demonstrated that a decrease in FLJ13639 mRNA corresponds to a decrease in FLJ13639 protein production. In particular, FIG. 16 provides a photographic representation of a Western blot using an FLJ13639/P1-TAT (described in Example 6) mAb we developed and its use to detect FLJ13639/P1-TAT recombinant protein (left part of the blot) as well as one ALL cell line (OM9;22) and the control lymphoblatoid cell line EBV-LIN. A single band is observed corresponding to the size of a dimer, but the difference in level of protein expression in the acute leukemia line versus the control line reflects the difference detected by RT-PCR, which demonstrates that a decrease in FLJ13639 mRNA results in a decrease in FLJ1363 protein expression.

### EXAMPLE 5

This Example demonstrates that, consistent with the cell line results shown in Example 4, a decreased expression of FLJ13639 is correlated with increased expression of CD24 in biological samples obtained from cancer patients. This Example also demonstrates that a high CD24/low FLJ13639P1 ratio is predictive of poor survival in leukemia patients.

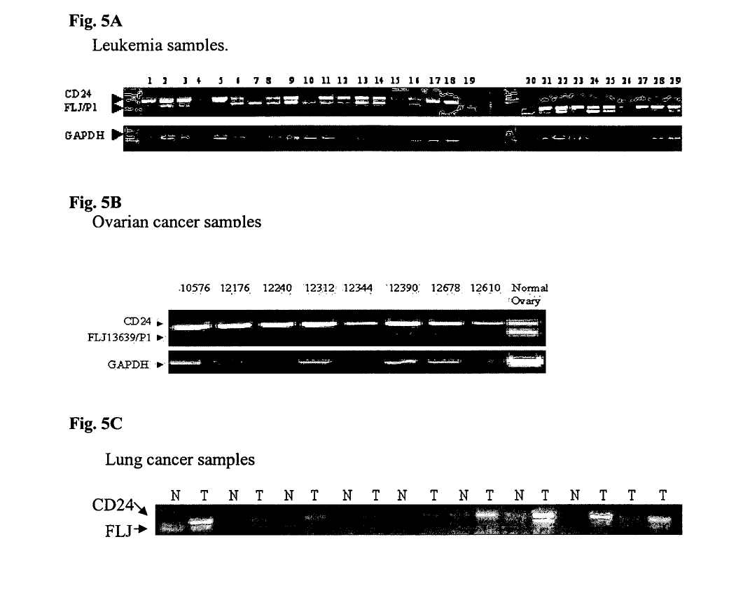

To obtain the data presented in FIGS. 5A-5C, we performed RT-PCR assay on a series of patient samples from acute leukemia, ovarian and lung cancer. All 10 highly invasive ovarian tumors showed a high CD24/low FLJ13639P1 profile (FIG. 5B) as well as the 10 lung tumors studied (FIG. 5C), as opposed to the corresponding adjacent tissue that showed a low CD24/high FLJ13639P1 profile (T=tumor, N=adjacent normal tissue). In acute leukemia, we performed the CD24/FLJ13639 RT-PCR assay on 29 diagnostic samples. Half of the samples showed a high CD24/low FLJ13639P1 (FIG. 5A).

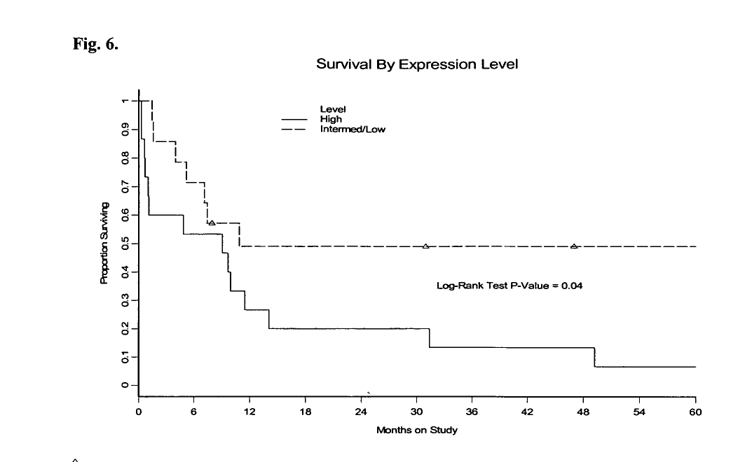

As shown in FIG. 6, a Kaplan-Meier survival curve based on the classification of the 29 patient samples demonstrates that the samples presenting a high CD24/low FLJ13639/P1 profile had a statistically significant lower survival rate that the intermediate/Low group (samples that presented a low CD24/high FLJ13639/P1 as well as samples where CD24 and FLJ13639/P1 levels gave a ratio close to 1 (Intermediate)). The Log-Rank test provided us with a P-Value that is significant (p=0.04).

### EXAMPLE 6

This Example demonstrates that the ratio of expression levels of CD24 to FLJ13639 is an independent prognostic factor in breast cancer.

### Tissues

Tissue microarrays (TMAs) were constructed from an unselected cohort of formalin fixed, paraffin embedded breast carcinomas treated at Roswell Park Cancer Institute between 1993 and 2001, after appropriate Institutional Review Board approval. Two 1 mm cores were transferred from each tumor to recipient blocks.

Procurement of tissue used for molecular studies has was performed according to conventional techniques (see, i.e., Varma G, et al. Cancer (2005) 19; 93(6):699-708). Fresh samples of breast cancer were obtained from patients from Western New York and Belarus, after appropriate Institutional Review Board approval. Patients with a strong family history of breast cancer or known to carry BRCA mutations were excluded. All samples were processed at RPCI. Samples from Belarus were collected between August 2002 and January 2003, snap frozen and transported on dry ice to the United States. Data on these patients was limited to clinical and pathological description of the disease at time of treatment (Varma G, et al. Cancer. 2005 Sep. 19; 93(6):699-708). RNA quantification demonstrated that 3 samples were degraded. Western New York samples were obtained from the tissue procurement facility at Roswell Park Cancer Institute (RPCI). Data was unavailable for two patients.

RNA Extraction from Solid Tumors

Fresh tissue samples frozen in liquid nitrogen and stored at −80° C. were homogenized using a Tekmar Tissumizer (Tekmar, Cincinnati, Ohio). Total RNA isolation was performed with Trizol reagent (Invitrogen, Carlsbad, Calif.), as per manufacturer's protocol. RNA quality and quantity were determined by an Agilent 2100 bioanalyzer (Agilent Technologies, Palo Alto, Calif.) as well as spectrophotometry.

### Reverse Transcription and Polymerase Chain Reaction

Reverse transcription was done in 20 μl containing a minimum of 1.25 μg of total RNA, 1 μl of 10 mM of deoxynucleotide triphosphate (NEB, Ipswitch, Mass.), 1 μl of 0.5 μg/μl oligo (dT) 18 (IDT, Coralville, Iowa), 1 μl of 40 U/μl ribonuclease inhibitor (Fermentas, Hanover, Md.), 2 μl of 10×reaction buffer (NEB), and 0.25 μl of 200 U/μl MMLV reverse transcriptase (NEB). After incubation with 1 μl of 5U μl RNase H (NEB) for 20 minutes, multiplex PCR reaction was carried out, allowing for simultaneous detection of CD24 and FLJ13639 expression levels. The primers are: CD 24 (resulting in an amplification product of 300 pb) forward: 5′TCTCTTCGTGGTCTCACTCT3′ (SEQ ID NO:12), reverse: 5′GATGTTGCCTCTCCTTCATC3′ (SEQ ID NO:13). The primers for FLJ13639 P1 (resulting in an amplification product of 233 pb) are the same as those described in Example 1. The reactions were of an initial denaturation at 95° C. for 10 minutes, followed by 35 cycles: denaturation, 50 seconds at 95° C., annealing 50 seconds at 54° C. and extension 60 seconds at 75° C. Actin amplification was performed as an internal control, according to the following protocol: 15 seconds at 95° C., 30 seconds at 60° C. and 60 seconds at 75° C.

### Tissue Microarray Immunohistochemistry

After antigen retrieval, five μm TMA sections were reacted with an anti-CD24 mouse monoclonal antibody (Clone ML5—Pharmingen, San Diego, Calif.) at 1 μg/ml for 2 hours. The signals were detected using the mouse Envision kit (Dako, Carpentaria, Calif.). Diaminobenzidine (DAB) was used as chromogen, and hematoxylin as counterstain. CD24-positive breast cancer cells were included as external positive controls. Nuclear and cytoplasmic staining intensity were separately scored as 0 (absent), 1+ (weak), 2+ (moderate), and 3+ (strong).

### Statistical Analysis

Statistical analysis was performed using SAS software package, version 9.1 (Cary, N.C.). Pearson correlation coefficient, logistic, cumulative logistic, and Poisson models were used to assess the statistical significance of associations between CD24, FLJ13639 and CD24/FLJ13639 and clinicopathologic variables respectively. Univariate survival analysis was done according to Kaplan-Meier method; the difference in survival curves was assessed with the log-rank test. Multivariate survival analysis was done using the Cox proportional hazard model. The fitted Cox proportional hazard model was selected by backward selection with the level of stay=0.1, using variables that were found not to violate the proportional hazard assumption based on visual evaluation of log (−log (survival)) plot. Hazard Ratios and 95% confidence intervals were calculate based on parameter estimates from the fitted model.

### Results

The study population for evaluation of CD24 included 154 patients. The average age of the patient population was 56.9 years. Average tumor size was 2.8 cm, and 62 patients had stage IIB disease or greater. Sixty-five cases had positive lymph nodes with an average of 5.7 positive nodes per patient. Breast conserving surgery was performed on 95 patients. Adjuvant cytotoxic therapy was administered to 147 patients. Endocrine therapy was administered to 108 patients of a total of 116 with hormone receptor positive cancer.

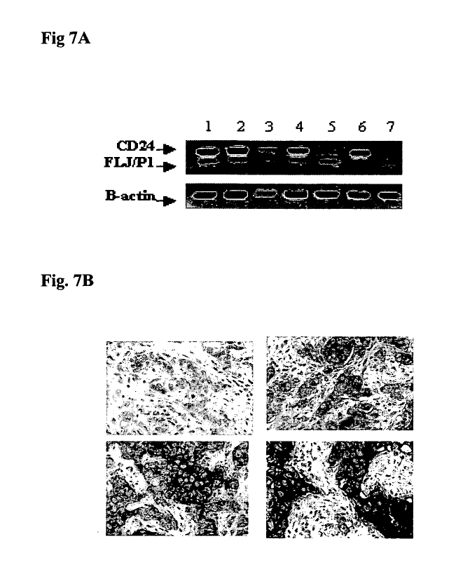

Tumors were scored as 3+ in 38 samples (24.7%) for cytoplasmic location and 37 samples (24.0%) for nuclear location. Only cases of intense CD24 staining (scored as 3+) were considered to have an abnormal expression level (FIG. 7B).

The median follow-up was 4.81 years. There were 25 recurrences and 18 cancer-related deaths. Nuclear localization of CD24 did not correlate with any clinical information. Intense cytoplasmic staining (3+) had a statistically significant association with disease stage (p=0.007), and hormone receptor status (ER p=0.03, PR p=0.04). There was no statistically significant association with necrosis, lymphovascular invasion, grade and nodal status.

### Molecular Analysis of Human Breast Cancer Samples and Clinical Correlation

Based on these IHC results regarding the clinical relevance of abnormally expressed CD24, we analyzed whether breast tumors over-expressing CD24 were in fact low or negative for FLJ13639 and, as a consequence, and whether high CD24/low/absent FLJ13639 molecular profile was associated with worse outcome. Multiplex RT-PCR was performed on 40 predominantly premenopausal breast cancers, 8 of which were from Belarus, and the rest from RPCI. Data on the Belarus patients was limited to clinical and pathological description of the disease at time of treatment and did not include follow up information. RNA quantification demonstrated that 3 of these samples were degraded. Clinical information was unavailable for two samples from RPCI. These 5 cases have been excluded from analysis.

The median age at diagnosis was 43 (range 29 to 56). Average tumor size was 3.9 cm and patients most commonly had stage IIB disease. Twenty-eight patients (80%) had node positive disease with an average of 2 positive nodes per patient. Twenty-four patients received radiation therapy. All American patients also received systemic chemotherapy, 2 for metastatic disease and 5 as a neoadjuvant modality. Hormone receptor status was not available for Belarus patients. Of the American women with receptor-positive disease, only one was not treated with anti-hormonal therapy. At a median follow up time of 42.77 months, 8 recurrences were documented and 9 of the American patients had died of cancer-related causes.

Multiplex RT-PCR results were assessed semi-quantitatively, with appropriate internal controls (FIG. 7A). Molecular expression data was then expressed as a ratio of CD24 over FLJ13639. Higher numerical values corresponded to the pathological molecular expression profile of high CD24 and low/absent FLJ13639. A threshold ratio of 3 was used to define patients with a less favorable molecular profile. Fourteen of the 35 samples (40.0%) available for analysis had a CD24/FLJ13639 ratio of 3 or greater. There was a statistically significant association between CD24 and FLJ13639 expression (p=0.01). CD24 had a positive correlation with tumor size (p=0.03), histological grade (p=0.04) and number of positive nodes (p<0.0001). FLJ13639 had a statistically significant correlation with estrogen receptor (ER) and progesterone receptor (PR) status (p values 0.02 and 0.03 respectively). Its expression had a negative correlation with nuclear grade (p=0.009) and number of positive nodes (p<0.0002). The ratio between CD24 and FLJ13639 (which defines the “at risk” profile) was a statistically significant predictor of decreased overall survival, with a hazard ratio of 1.3 at univariate analysis (p=0.03) (95% CI: 1.0-1.6). After adjusting for other factors, the estimated hazard ratio estimated by multivariate analysis for an increased CD24/FLJ13639 was 1.51 (p=0.005) (95% CI: 1.1-2.0). When CD24/FLJ13639 ratios were categorized using the value of 3 as a threshold to define increased risk, the ability to predict worse outcome became even stronger 3.65 at univariate analysis (p=0.03), with a hazard ratio of 8.9 at multivariate analysis (p=0.01).

### EXAMPLE 6

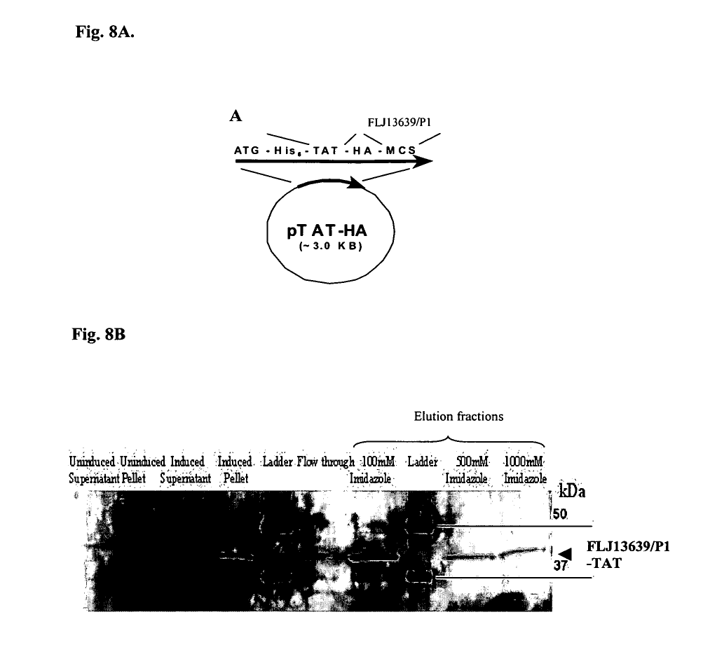

This Example demonstrates the generation of a recombinant FLJ13639/P1 protein. To produce this protein, we subcloned the FLJ13639/P1 cDNA in frame with the TAT protein transduction domain from the HIV virus. Briefly, the full length FLJ13639/P1 cDNA was amplified by PCR excluding the FLJ13639/P1 codon START. It was then subcloned within the TAT vector, which is depicted in FIG. 8A. This vector contains its own START codon, a 6×Histidine tag to be used for purification and the FLJ13639/P1 DNA sequence encoding the FLJ13639/P1 protein. This allows the recombinant protein to be produced in vitro in bacteria and to be subsequently purified making use of the 6×HIS tag on Nickel columns. The amino acid sequence of the P1-TAT-recombinant proteins can be used for in vivo protein import in cells with a high efficiency (16) as compared with retrovirus. In addition, this approach potentially allows analysis of the dosage of protein intake by the cells.

We successfully expressed the recombinant protein in BL21 bacteria and further purified the protein using NiNTA columns (using the binding capacity of a 6×His tag located before the TAT tag) (FIG. 8B.). This recombinant protein is referred to herein alternatively as “P1-TAT” and “FLJ13639/P1-TAT.” The amino acid sequence of P1-TAT is provide as SEQ ID NO:5. The sequence of the DNA encoding P1-TAT is provided as SEQ ID NO:14. FLJ13639 P1 was cloned into the expression vector using the forward primer having the sequence 5′CTCGAGTCCTGTACCGCAGCG (SEQ ID NO:15) and the reverse primer having the sequence 5′GAATTCTCCTATTTAAATGTTCGA (SEQ ID NO:16). The P1-TAT protein is soluble in buffer containing salts and glycerol, and is suitable for administration by injection following high-grade purification.

### EXAMPLE 7

This Example demonstrates that administering P1-TAT can regulate the expression of CD24 in leukemia and lung cancer cells.

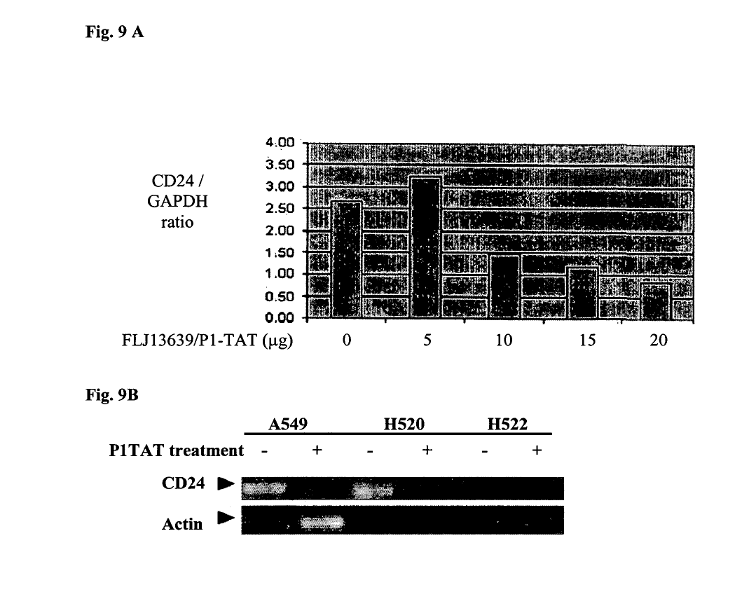

Different leukemia cell lines were incubated with the P1-TAT recombinant protein to assess the effect of P1 function on CD24 expression. Specifically, the UoCB1 cell line was incubated with increasing amount of P1-TAT recombinant protein. After 48 hours of incubation, cells were harvested, RNA was extracted and cDNA was used for CD24 expression assessment by RT-PCR. The CD24 expression levels were inversely proportional to the amount of P1-TAT protein added to the cultures (FIG. 9A). Additional cell lines (OM9:22, ALL-1. K562, 207) were assayed for CD24 expression upon incubation with 20 μg of P1-TAT and showed a marked CD24 down-regulation (data not shown). Further, the same type of assays were performed on lung cancer cells with similar results (FIG. 9B).

### EXAMPLE 8

This Example demonstrates that administering P1-TAT can inhibit an invasive phenotype in leukemia cells.

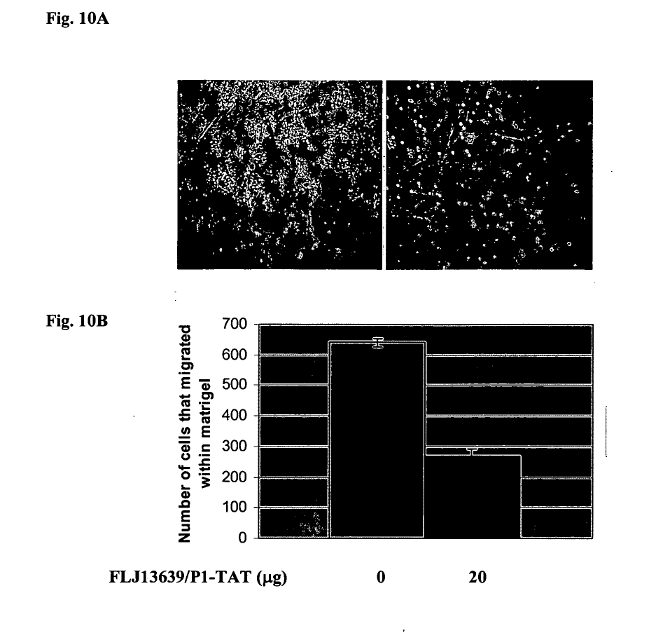

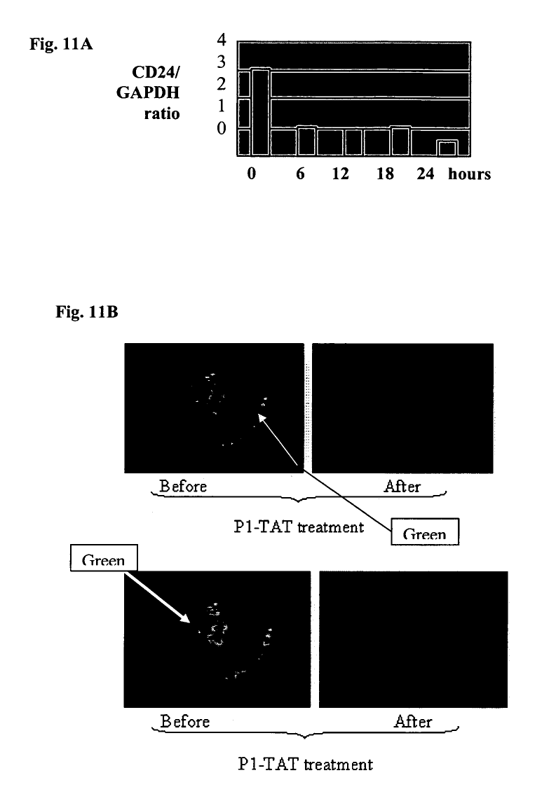

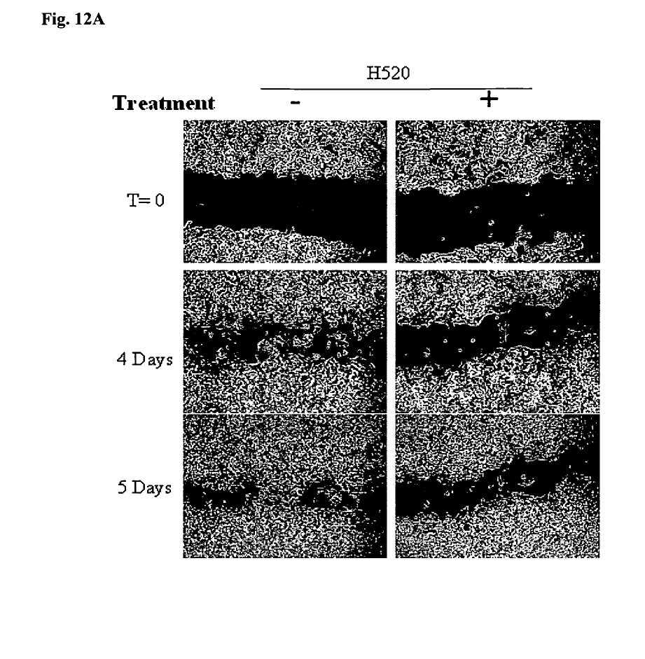

The UoCB1 cell line was assayed for alteration of its invasiveness potential upon treatment with the P1-TAT recombinant protein. The cells were treated with 20 μg of P1-TAT for 48 h and assayed using a standard Matrigel invasion assay (treatment versus no treatment). To perform the Matrigel invasion assay, cells were deposited on top of a matrigel well and chemoattractant (fetal bovine serum) solutions were positioned below the matrigel matrix. Only cells with an invasive potential will “travel” toward the chemoattractant solutions through the matrigel. The results from this assay showed that the treatment with P1-TAT decreased the invasion potential of UoCB1 cells by a 2.4 fold factor (FIG. 10A).

### EXAMPLE 9

This Example demonstrates that administering exogenous P1-TAT can downregulate the expression of CD24 in leukemia and ovarian cancer cells.

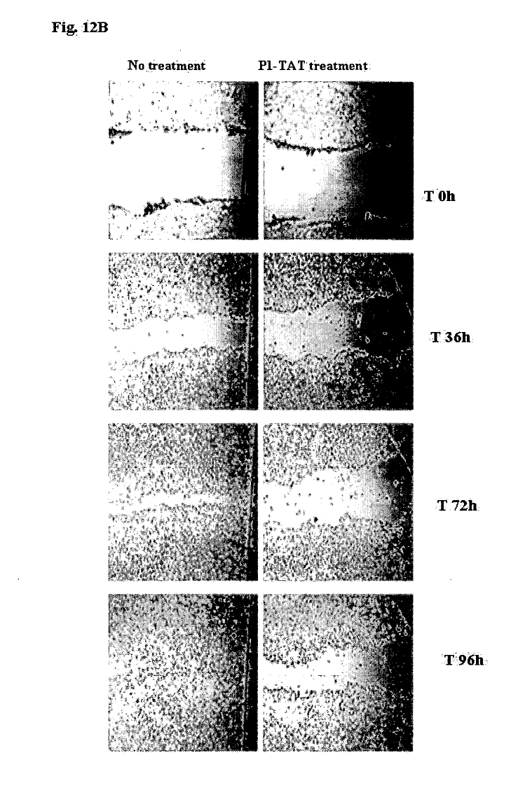

A time-course incubation (i.e. incubation of the cell lines with 20 ug of P1-TAT for 0, 6, 12, 16, 24 and 48 hours) was performed with 20 μg of P1-TAT to assess the time frame for CD24 down-regulation after incubation with the recombinant protein. CD24 levels were significantly downregulated six hours after incubation with P1-TAT (FIG. 11A). In addition, CD24 protein expression was down-regulated after 48 hours of treatment with P1-TAT (FIG. 11B). The same results were obtained using the OVCAR3 ovarian cancer cell line (not shown).

### EXAMPLE 10

This Example demonstrates that administering P1-TAT can inhibit an invasive phenotype in lung cancer and breast cancer cells and inhibit their proliferation. To obtain the data presented in this Example, lung cancer cell lines (FIG. 12A) (H520) and MCF7 (FIG. 12B) were incubated (or not incubated, as a control) with P1-TAT in a wound healing assay. This was performed by growing the cells to confluence and forming a “wound” by scoring the culture with a pipette tip. The cells were then treated with P1-TAT and assessed for their ability to close the gap between the two artificially created cell populations. As can be seen from FIGS. 12A and 12B, the P1-TAT treatment significantly reduced the motility of the cancer cells, which demonstrates that their invasion potential was greatly diminished and that their proliferation was inhibited.

### EXAMPLE 11

This Example demonstrates that administrating P1-TAT restores aerobic respiration by shifting energy metabolism from anaerobic glycolysis to aerobic respiration.

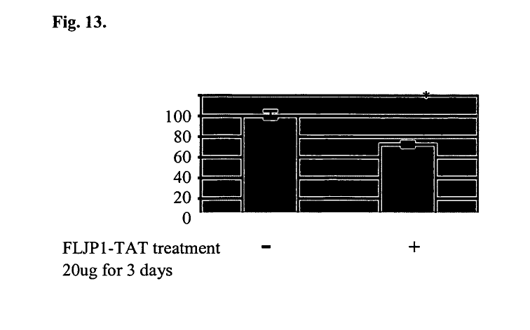

Mitochondria are well known for their essential role in cell respiration and apoptosis. Cancer cells are known to have altered mitochondrial respiration, where pyruvate is catabolized in the lactic acid fermentation pathway instead of being used in the Krebbs cycle in the mitochondria, which produces energy far more efficiently. HeLa cells have been shown to produce relatively large quantity of lactic acid and we have demonstrated they exhibit a high CD24/low FLJ13639/P1 profile (not shown). In this Example, HeLa cells were treated (or not treated, as a control) with P1-TAT recombinant protein for 3 days. Culture medium was tested for lactic acid levels. A significantly lower level of lactic acid was observed upon treatment with the P1-TAT recombinant protein (FIG. 13).

### EXAMPLE 12

This Example demonstrates that administration of exogenous P1-TAT can enhance the activity of a chemotherapeutic agent on chemoresistant cancer cells.

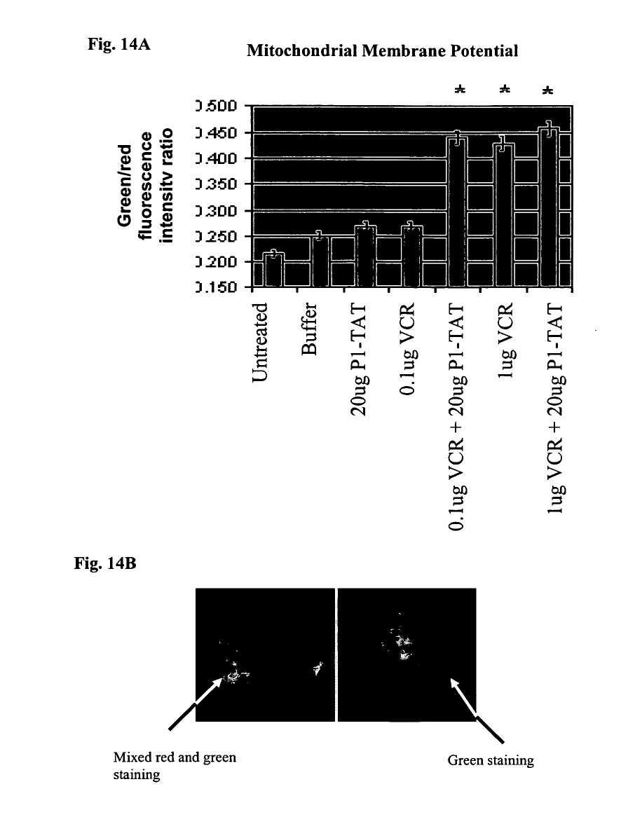

Disruption of the mitochondrial membrane potential (Δψₘ) is an early event associated with apoptosis and may be one of several factors responsible for cytochrome c release (17, 18). Using JC-1 mitochondrial potential sensor (Molecular Probes, Inc) staining and fluorescence microscopy, we analyzed the Δψₘ in VCR (vincristin) treated HL60 cells. In healthy cells, the dye accumulates and aggregates in mitochondria, where it fluoresces bright red. In apoptotic cells, however, it cannot enter mitochondria if the Δψₘ is altered and fluoresces bright green as a cytoplasmic monomer. Therefore, mitochondria depolarization is indicated by a decrease of the red/green fluorescence intensity ratio. We observed that untreated HL60 cells as well as cells treated with protein buffer, 20 ug of FLJP 1-TAT or 0.1 ug of VCR showed a low green/red fluorescence ratio. However, upon treatment with 0.1 ug of VCR+20 ug of P1-TAT, 1 ug of VCR or 1 ug of VCR+20 ug of P1-TAT, a significant increase of the green/red ratio (p<0.01) is detected (FIGS. 14A and B).

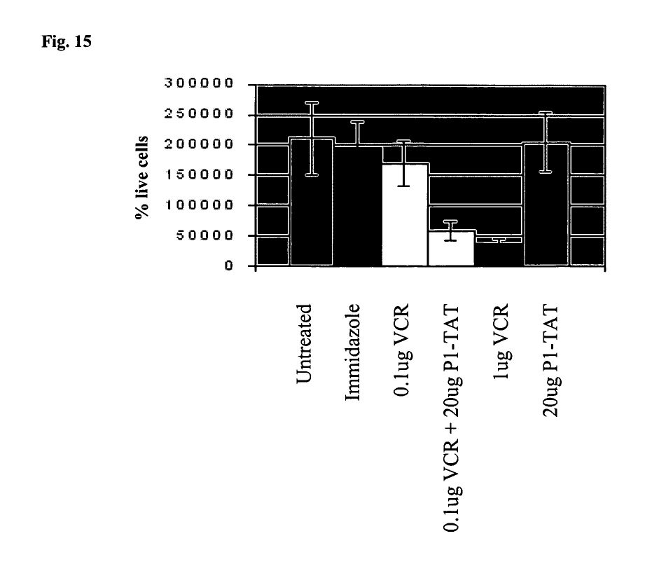

To assess whether FLJ13639/P1 restoration would affect chemosensitivity of low P1-TAT (high CD24) cells, HL60/VCR (Vincristin resistant) cells were incubated with different combinations of vincristin and FLJ13639/P1-TAT protein, as well as appropriate controls (FIG. 15). Viability assays by conventional MTT assay protocol (Promega) were performed at 48 h after the beginning of the experiment. Vincristin (0.1 μM) had almost no effect on the viability of the HL60 cells, Vincristin (1 μM) reduced cell viability by 90%. The combination of 0.1 μM and 20 μg of FLJ13639/P1-TAT had the same effect as the incubation with a 10× concentrated solution of vincristin (FIG. 15).

The invention has been described through specific embodiments. However, routine modifications to the compositions, methods and devices will be apparent to those skilled in the art and such modifications are intended to be covered within the scope of the invention.

### REFERENCES

1—Coignet et al, Gene Chrom Cancer 1999 25:222-9
2—Zojer et al, Blood 2000 95:1925-30
3—Shaughnessy et al, Blood 2000 96:1505-11
4—Avet-Loiseau et al, Blood 2002 99:2185-91
5—Avet-Loiseau et al, Blood 1999 94:2583-9
6—Cuneo et al, Blood 1999 93:1372-80
7—Meyer et al, Leukemia 2001 15(9): 1471-4.
8—Rabbits, Nature 372:143-149, 1994
9—Sawyer, Lancet 349:196-199, 1997
10—Knudson, PNAS USA 68:820-823
11—Jornvall et al. Biochemistry. 1995 May 9; 34(18):6003-13.
12—Scheurer et al. Proteomics. 2004 June; 4(6):1737-60.
13—Raife et al. Am J Clin Pathol. 1994 March; 101(3):296-9.
14—Lavabre-Bertrand et al. Leukemia. 1994 March; 8(3):402-8.
15—Kristiansen et al. J Mol Histol. 2004 March; 35(3):255-62
16—Becker-Hapak et al. Methods. 2001 July; 24(3):247-56.
17—Kroemer et al. Immunol Today. 1997 January; 18(1):44-51.
18—Heiskanen et al. J Biol Chem. 1999 Feb. 26; 274(9):5654-8.

We claim:

1. A method of determining the prognosis of an individual diagnosed with or suspected of having cancer comprising:
i) obtaining a biological sample from the individual;
ii) measuring the amount of CD24 mRNA or CD24 protein in the sample;
iii) measuring the amount of FLJ13639 mRNA or FLJ13639 protein in the sample;
iv) comparing the amount of CD24 mRNA to the amount FLJ13639 mRNA, or comparing the amount CD24 protein to the amount of FLJ13639 protein to obtain a CD24 to FLJ13639 expression ratio;
wherein a CD24 to FLJ13639 expression ratio of 3:1 or greater is indicative of an unfavorable prognosis for the individual.

2. The method of claim 1, wherein the amount of CD24 mRNA and the amount of FLJ13639 mRNA are determined by performing RT-PCR or by microarray analysis.

3. The method of claim 2, wherein performing RT-PCR of FLJ13639 mRNA amplifies a cDNA comprising the sequence of SEQ ID NO: 1 and/or wherein performing RT-PCR of the CD24 mRNA amplifies a cDNA comprising the sequence of SEQ ID NO:6.

4. The method of claim 1, wherein the amount of CD24 protein and the amount of FLJ13639 protein are determined by Western blotting, flow cytometry, immunhistochemistry, or ELISA.

5. The method of claim 1, wherein the biological sample is blood, bone marrow or tumor.

6. The method of claim 1, wherein the individual has been diagnosed with or is suspected of having a cancer selected from prostate cancer, breast cancer, colorectal cancer, lung cancer (including small cell and non-small cell lung cancer), ovarian cancer, melanoma, urinary system cancer, uterine cancer, endometrial cancer, pancreatic cancer, oral cavity cancer, thyroid cancer, stomach cancer, brain cancer, hematological cancers, and combinations thereof.

7. The method of claim 5, wherein the hematological cancer is selected from Hodgkins Lymphoma, Non-Hodgkins Lymphoma (NHL), leukemias and myelomas.

8. The method of claim 1, wherein an unfavorable prognosis for the individual identifies the individual as a candidate for therapy with recombinant FLJ13639 protein.

9. An isolated, purified protein comprising the sequence of SEQ ID NO:2.

10. The protein of claim 9, wherein the protein is a recombinant protein.

11. The protein of claim 10, further comprising a protein purification tag and/or a protein transduction domain.

12. The protein of claim 10, wherein the protein comprises the amino acid sequence of SEQ ID NO:5.

13. A method for improving the prognosis of an individual diagnosed with or suspected of having cancer comprising administering to the individual a composition comprising FLJ13639 protein in an amount effective to improve the prognosis of the individual.

14. The method of claim 13, wherein the composition comprises a pharmaceutically acceptable carrier.

15. The method of claim 13, wherein the administration of the FLJ13639 protein inhibits the growth of cancer cells in the individual.

16. The method of claim 13, wherein the administration of the FLJ13639 protein inhibits metastasis of cancer cells in the individual.

17. The method of claim 13, wherein the FLJ13639 protein comprises the sequence of SEQ ID NO:2.

18. The method of claim 13, further comprising administering a chemotherapeutic agent to the individual, wherein administration of the composition comprising the FLJ13639 protein enhances the activity of the chemotherapeutic agent.

19. The method of claim 18, wherein the chemotherapeutic agent is selected from the group consisting of vincristin, doxorubicin and cisplatin.

20. The method of claim 13, wherein the individual has been diagnosed with or is suspected of having a cancer selected from prostate cancer, breast cancer, colorectal cancer, lung cancer (including small cell and non-small cell lung cancer), ovarian cancer, melanoma, urinary system cancer, uterine cancer, endometrial cancer, pancreatic cancer, oral cavity cancer, thyroid cancer, stomach cancer, brain cancer, hematological cancers, and combinations thereof.
